(19) United States
(12) Patent Application Publication
(10) Pub. No.: US 2024/0103019 A1
(43) Pub. Date: Mar. 28, 2024

(54) USE OF THE EMM ANTIGEN AS A BIOMARKER OF INHERITED GPI DEFICIENCIES

(71) Applicants: INSTITUT NATIONAL DE LA SANTÉ ET DE LA RECHERCHE MÉDICALE, Paris (FR); ASSISTANCE PUBLIQUE - HÔPITAUX DE PARIS, Paris (FR); CENTRE NATIONAL DE LA RECHERCHE SCIENTIFIQUE (CNRS), Paris (FR); FONDATION IMAGINE, Paris (FR); UNIVERSITÉ PARIS CITÉ, Paris (FR); UNIVERSITE ANTILLES GUYANE, Pointe a Pitre (FR); ETABLISSEMENT FRAN¿AIS DU SANG (EFS), La Plaine Saint Denis (FR)

(72) Inventors: Thierry PEYRARD, Paris (FR); Slim AZOUZI, Paris (FR); Oliver HERMINE, Paris (FR); Yves COLIN-ARONOVICZ, Paris (FR); Romain DUVAL, Paris (FR); Caroline LE VAN KIM, Paris (FR)

(21) Appl. No.: 18/256,453

(22) PCT Filed: Dec. 8, 2021
(86) PCT No.: PCT/EP2021/084699

§ 371 Date: Jun. 8, 2023

(30) Foreign Application Priority Data
Dec. 9, 2020 (EP) 20306520.6

(51) Int. Cl. G01N 33/68; G01N 33/80

(52) U.S. Cl. CPC G01N 33/6893; G01N 33/80; G01N 2405/06; G01N 2440/38; G01N 2800/2857; G01N 2800/34

(57) ABSTRACT

Glycosylphosphatidylinositol (GPI) is a glycolipid that anchors more than 150 proteins to the cell surface. Pathogenic variants in several genes that participate in GPI biosynthesis cause inherited GPI deficiency (IGD) disorders. Here, the inventors reported that homozygous null alleles of PIGG, a gene involved in GPI modification, are responsible for the rare Emm-negative blood phenotype. Using a panel of K562 cells defective in both the GPI-transamidase and GPI remodeling pathways, they demonstrate that the Emm antigen, whose molecular basis has remained unknown for decades, is carried only by free GPI and that its epitope is composed of the second and third ethanolamine of the GPI backbone. Importantly, the inventors show that the decrease in Emm expression in several IGD patients is indicative of GPI defects. Overall, our findings establish Emm as a novel blood group system and have important implications for understanding the biological function of human free GPI.

### FIELD OF THE INVENTION

The present invention is in the field of medicine.

### BACKGROUND OF THE INVENTION

The Emm-negative rare blood phenotype was first described in 1973 but remains one of the last blood types with an unknown genetic basis (Emm currently included in the 901 series of high-prevalence red cell antigens). Anti-Emm is rare and seems to be a naturally-occurring antibody, as six of the reported anti-Emm antibodies were found in non-transfused males⁻¹. The Emm-negative phenotype is thought to be inherited as a recessive trait¹. The exact nature of the Emm antigen has so far remained a mystery, but it has been proposed that the Emm antigen is carried on a glycosylphosphatidylinositol (GPI)-anchored protein (GPI-AP) in the red blood cell (RBC) membrane since its expression is strongly decreased in RBCs from paroxysmal nocturnal hemoglobinuria (PNH) patients². PNH is an acquired GPI deficiency caused by somatic mutations in the PIGA gene³, except in several patients in whom it is caused by combinations of somatic and heterozygous germline mutations in the PIGT gene^{4,5}. GPI is a glycolipid that tethers more than 150 different proteins to the cell surface including the complement-inhibitory glycoprotein (CD59), and at least 22 genes termed phosphatidyl inositol glycan (PIG) genes, including PIGA and PIGT, which are involved in the synthesis of GPI-APs⁶. GPI-AP anchoring is a multistep process that includes synthesis of the GPI precursor in the endoplasmic reticulum (ER), protein attachment to GPI, and remodeling of the GPI-AP complex in the ER and Golgi⁷⁻⁹. It is also known that some of the GPI molecules synthesized in the ER are transported to the cell surface and expressed as free, unlinked GPIs¹⁰. To date, germline mutations in PIG/PGAP (Post GPI Attachment to proteins) genes have been reported to cause various inherited GPI deficiency (IGD) disorders and phenotypes, including multiple congenital anomalies-hypotonia-seizures syndrome (MCAHS), and hyperphosphatasia with mental retardation syndrome (HPMRS)/Mabry syndrome¹¹.

### SUMMARY OF THE INVENTION

The present invention is defined by the claims. In particular, the present invention relates to the use of the Emm antigen as a biomarker of Inherited GPI deficiencies.

### DETAILED DESCRIPTION OF THE INVENTION

Glycosylphosphatidylinositol (GPI) is a glycolipid that anchors more than 150 proteins to the cell surface. Pathogenic variants in several genes that participate in GPI biosynthesis cause inherited GPI deficiency (IGD) disorders. Here, the inventors reported that homozygous null alleles of PIGG, a gene involved in GPI modification, are responsible for the rare Emm-negative blood phenotype. Using a panel of K562 cells defective in both the GPI-transamidase and GPI remodeling pathways, they demonstrate that the Emm antigen, whose molecular basis has remained unknown for decades, is carried only by free GPI and that its epitope is composed of the second and third ethanolamine of the GPI backbone. Importantly, the inventors show that the decrease in Emm expression in several IGD patients is indicative of GPI defects. Overall, our findings establish Emm as a novel blood group system and have important implications for understanding the biological function of human free GPI.

Accordingly the present invention relates to the use of the Emm antigen as a biomarker of inherited glycosylphosphatidylinositol (GPI) deficiencies.

In particular, the present invention relates to a method of diagnosing an inherited glycosylphosphatidylinositol (GPI) deficiency in a subject comprising detecting the expression of the Emm antigen in a sample of red blood cells obtained from the subjecting wherein detecting an alteration of the expression of Emm antigen indicates that the subject suffers from an inherited GPI deficiency.

As used herein, the term “glycosylphosphatidylinositol” or “GPI” has its general meaning in the art and refers to a glycolipid that anchors more than 150 proteins to the cell surface, and these proteins, termed “GPI-anchored proteins” or “GPI-Aps”, perform a variety of functions as enzymes, adhesion molecules, complement regulators, and coreceptors in signal transduction pathways. Reduced surface levels of GPI-APs or abnormal GPI-AP structure can therefore result in variable manifestations that include glycosylphosphatidylinositol deficiencies.

As used herein, the term “inherited glycosylphosphatidylinositol deficiency” or “inherited GPI deficiency” relates to a group of clinically and genetically heterogeneous conditions characterized by reduced surface levels of GPI-APs or abnormal GPI-AP structures. The term is also called “glycosylphosphatidylinositol biosynthesis defects” or “GPIBDs”. While complete GPI deficiency is lethal, several disorders involving the GPI machinery have been identified since 2006, and are thought to be caused by hypomorphic mutations (see e.g. Chiyonobu, T., Inoue, N., Morimoto, M., Kinoshita, T., & Murakami, Y. (2014). Glycosylphosphatidylinositol (GPI) anchor deficiency caused by mutations in PIGW is associated with West syndrome and hyperphosphatasia with mental retardation syndrome. Journal of Medical Genetics, 51(3), 203-207). For said disorders, the phenotype is characterized by hyperphosphatasia and intellectual disability (ID), and these disorders are often called “hyperphosphatasia with intellectual disability (formerly ‘mental retardation’) syndrome” or Mabry syndrome. In particular, the method of the present invention is particularly suitable for diagnosing inherited GPI deficiencies characterized by intellectual disability and/or epilepsy.

In particular, the method of the present invention is particularly suitable for diagnosing an inherited GPI deficiency when the expression of GPI-anchored proteins is however decreased in the red blood cells of the subject. More particularly, the method of the present invention is particular suitable for diagnosing an inherited GPI deficiency when the expression of GPI-APs (eg. CD59, CD55, FLAER, CD16, etc. . . . ) in the blood cells does not reflect the GPI defect of the subject.

In particular, the method of the present invention is also particularly suitable for diagnosing a paroxysmal nocturnal hemoglobinuria.

As used herein, the term “red blood cell” or “RBC” also known as erythrocytes are highly-specialized cells responsible for delivery of oxygen to, and removal of carbon dioxide from, metabolically-active cells via the capillary network. They are shaped as biconcave discs and average about 8-10 microns in diameter. Several phenotypic markers of RBC have been described an typically include CD46, CD55, CD100, CD175s, CD117; CD29, CD31, CD35, CD36, CD44, CD45RB, CD47, CD59, CD81, CD99, CD108, CD147, CD164, CD222, CD235a, CD90, CD105, Monocarboxylate transporter 1 (MCT1) and CD233. More particularly phenotypic markers of mature RBCs include CD235a, Band3, RH proteins, GYPC/D, CD59, CD55, CD108, CD44, CD47, Glut1, and MCT1.

A used herein, the term “Emm antigen” has its general meaning in the art and refers to the antigen described in Daniels G L, Taliano V, Klein M T, McCreary J. Emm. A red cell antigen of very high frequency. Transfusion. 1987; 27(4):319-321. In particular, as demonstrated in the EXAMPLE, its epitope is composed of the second and third ethanolamine of the free GPI backbone. Several anti-Emm antibodies have been described in the prior art (see e.g. Daniels G L, Taliano V, Klein M T, McCreary J. Emm. A red cell antigen of very high frequency. Transfusion. 1987; 27(4):319-321).

Typically the alteration of the expression of the Emm antigen consists of a decrease or an increase in said expression in comparison of a control reference level (e.g. measured in healthy individuals).

Methods for detecting the expression of Emm antigen on red blood cells are well known in the art and typically involved use of immunoassays such as described in the EXAMPLE. In particular, standard methods for detecting the expression of a specific surface marker such as Emm antigen at cell surface (i.e. red blood cell surface) are well known in the art. Typically, the step consisting of measuring the expression level of Emm at the red blood cell surface may consist in collecting a population of red blood cells from the subject and using at least one differential binding partner directed against the Emm antigen, wherein said red blood cells are bound by said binding partners to said Emm antigens.

As used herein, the term “binding partner directed against the Emm antigen” refers to any molecule (natural or not) that is able to bind the Emm antigen with high affinity. Said binding partners include but are not limited to antibodies, aptamer, and peptides.

The binding partners may be antibodies that may be polyclonal or monoclonal, preferably monoclonal, specifically directed against said Emm antigen. In some embodiments, the binding partners may be a set of aptamers. Polyclonal antibodies of the invention or a fragment thereof can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies of the invention or a fragment thereof can be prepared and isolated using any technique that provides for the production of antibody molecules by continuous cell lines in culture. Techniques for production and isolation include but are not limited to the hybridoma technique originally; the human B-cell hybridoma technique; and the EBV-hybridoma technique.

The binding partners of the invention such as antibodies or aptamers may be labelled with a detectable molecule or substance, such as preferentially a fluorescent molecule, or a radioactive molecule or any others labels known in the art. Labels are known in the art that generally provide (either directly or indirectly) a signal.

As used herein, the term “labelled”, with regard to the antibody or aptamer, is intended to encompass direct labelling of the antibody or aptamer by coupling (i.e., physically linking) a detectable substance, such as a fluorophore [e.g. fluorescein isothiocyanate (FITC) or phycoerythrin (PE) or Indocyanine (Cy5)]) or a radioactive agent to the antibody or aptamer, as well as indirect labelling of the probe or antibody by reactivity with a detectable substance.

Preferably, the antibodies against the Emm antigen are already conjugated to a fluorophore (e.g. FITC-conjugated and/or PE-conjugated). The aforementioned assays may involve the binding of the binding partners (i.e. antibodies or aptamers) to a solid support. The solid surface could a microtitration plate coated with antibodies against Emm antigen. After incubation of the red blood cell sample, red blood cells specifically bound to the Emm binding partner may be detected with an antibody to a common red blood cell marker. Alternatively, the solid surfaces may be beads, such as activated beads, magnetically responsive beads. Beads may be made of different materials, including but not limited to glass, plastic, polystyrene, and acrylic. In addition, the beads are preferably fluorescently labelled. In some embodiments, fluorescent beads are those contained in TruCount™ tubes, available from Becton Dickinson Biosciences, (San Jose, California).

According to the invention, methods of flow cytometry are preferred methods for measuring the level of Emm antigen at the red blood cell surface. Said methods are well known in the art. For example, fluorescence activated cell sorting (FACS) may be therefore used. Typically, a FACS method such as described in the Example here below may be used to measuring the level of Emm antigen at the surface of red blood cells.

When the subject is diagnosed with an inherited GPI deficiency then the physician can prescribe the most accurate treatment. In particular, the subject is administered with a HDCA inhibitor. As used herein, the term “HDAC inhibitor” refers to a molecule capable of inhibiting the deacetylase function of one or more HDACs. HDAC inhibitors are well known in the art and include, but are not limited to, trifluoroacetylthiophene-carboxamides, tubacin, tubastatin A, WT 161, PCI-34051, MC1568, MC1575, suberoylanilide hydroxamic acid (also known as vorinostat or SAHA), Trichostatin A, sodium butyrate, valproic acid, M344, Sriptaid, Trapoxin, Depsipeptide (also known as Romidepsin), MS275 (Entinostat), 4-phenylimidazole, MC1293, droxinostat, curcumin, belinostat (PXD101), panobinostat (LBH589), MGCD0103 (mocetinostat), parthenolide and givinostat (ITF2357). In particular, in case of seizures, the patient is administered with anti-epileptic drugs. As used herein, the term “anti-epileptic drug” or “AED” generally encompasses pharmacological agents that reduce the frequency or likelihood of a seizure. There are many drug classes that comprise the set of antiepileptic drugs (AEDs), and many different mechanisms of action are represented. For example, some medications are believed to increase the seizure threshold, thereby making the brain less likely to initiate a seizure. Other medications retard the spread of neural bursting activity and tend to prevent the propagation or spread of seizure activity. Some AEDs, such as the Benzodiazepines, act via the GABA receptor and globally suppress neural activity. However, other AEDs may act by modulating a neuronal calcium channel, a neuronal potassium channel, a neuronal NMDA channel, a neuronal AMPA channel, a neuronal metabotropic type channel, a neuronal sodium channel, and/or a neuronal kainite channel.

The invention will be further illustrated by the following figures and examples. However, these examples and figures should not be interpreted in any way as limiting the scope of the present invention.

### Example

Methods:

Subjects

Samples from three Emm-negative individuals (P1, P2 and P3), all with anti-Emm, were obtained from a cryopreserved rare reference material collection from the National Reference Center for Blood Group (CNRGS, Paris, France), approved by the local ethics committee (CPP Ile-de-France II) and the French Research Ministry (ref DC-2016-2872). IGD patients were recruited via Polyweb and came from the Necker Hospital (Paris, France) and Andre Mignot Hospital (Versailles, France). Informed consent was obtained from both the participants and their legal representatives. RBC samples from voluntary Etablissement Français du Sang (EFS) blood donors who had consented to the use of their blood for research purposes were used as Emm-positive controls.

Serological Testing

Standard hemagglutination tests were used for the assessment of anti-Emm reactivity and RBC typing. The anti-Emm was sourced from two unrelated men (P1 and P3) described in 1986 in the original paper¹. By indirect antiglobulin gel testing (ID-Card LISS/Coombs; DiaMed, Bio-Rad, headquartered in Hercules, CA, USA) at 22° C. on papain-treated RBCs, P1, P2, and P3 sera were found to react 3+(titer 8, score 35), 3+(titer 128, score 72), and 3+(titer 8, score 35) and, respectively. Anti-Emm eluates were prepared after adsorption of human polyclonal anti-Emm from the serum sample of P2 onto a pool of 3 group O papain-treated RBCs, followed by an acid elution test with the Gamma ELU-KIT II device (Immucor Inc. Norcross, GA, USA). The Emm specificity of the eluate was checked using Emm-positive and Emm-negative RBCs, as well as the absence of contaminating ABO antibodies. The investigation by flow cytometry of the antibody class and subclass of the eluate from P2 was consistent with an IgG3 antibody.

Whole-Exome Sequencing and Data Analysis

Genomic DNA was extracted from leukocytes using the MagnaPure system (Roche). Exome capture was performed with the Sure Select Human All Exon Kit (Agilent Technologies). Agilent Sure Select Human All Exon (58 Mb, V6) libraries were prepared from 3 μg of genomic DNA sheared with an ultrasonicator (Covaris) as recommended by the manufacturer. Barcoded exome libraries were pooled and sequenced with a HiSeq2500 system (Illumina Inc. San Diego, CA, USA), generating paired-end reads. After demultiplexing, sequences were mapped on the human genome reference (NCBI build 37, hg19 version) with Burrows-Wheeler Aligner (BWA). The mean depth of coverage obtained for the three probands' exome libraries was >120X with >=96% and >=94% of the targeted exonic bases covered by at least 15 and 30 independent sequencing reads (>=96% at 15X>=94% at 30X). Variant calling was carried out with the Genome Analysis Toolkit (GATK), SAMtools, and Picard tools. Single-nucleotide variants were called with GATK Unified Genotyper, whereas indels were called with the GATK IndelGenotyper_v2. All variants with a read coverage of 23% and a Phred-scaled quality of 20% were filtered out. All variants were annotated and filtered with PolyWeb, an in-house annotation software program.

Cell Culture

K562 cells were cultured in IMIDM, 25 mM HEPES and GlutaMAX (Gibco) supplemented with 10% decomplemented (56° C.—30 minutes) fetal bovine serum and antibiotics (100 units/mL penicillin and 100 μg/mL streptomycin) at 37° C. under a humidified atmosphere containing 5% CO₂. Authentication of our K562 cell line was performed by Eurofins MWG.

Flow Cytometry

Thawed RBCs or K562 cells were washed 3 times in PBS (Gibco) and then resuspended in low-ionic strength buffer supplemented with 0.5% bovine serum albumin and incubated with anti-Emm eluate (1:2) or mouse monoclonal anti-CD59 (1:100; BD Pharmingen). Anti-Emm labeling was revealed with anti-human IgG-PE (1:100; Beckman Coulter), and anti-CD59 labeling was revealed with anti-mouse IgG-PE (1:100; Beckman Coulter). A FACSCantoII flow cytometer (BD Bioscience) and FlowJo software were used for data acquisition and analysis, respectively.

Immunofluorescence Confocal Microscopy

K562 cells were fixed and permeabilized using the Foxp3/Transcription Factor Staining Buffer Set (eBioscience™) according to the manufacturer's instructions. Briefly, 5×10⁶ cells were washed in PBS, incubated in 1×Fix/Perm buffer for 30 min at room temperature and washed twice in 1×Perm buffer. Cells were then incubated with anti-Emm antibody (1:2) in 1×Perm buffer for 120 min on ice. The cells were then washed and incubated with Alexa Fluor 488-conjugated goat anti-human IgG (Invitrogen) for 60 min on ice. After another set of washes in 1×Perm buffer, samples (0.1×10⁶ cells) were cytospun onto glass slides, and 10 μL of ProLong™ Diamond Antifade Mountant with DAPI (Molecular Probes) was added before the glass was sealed. Observation was performed using a 63×objective lens on a Zeiss LSM 700 laser scanning confocal microscope.

Western Blot

Cell lysates were prepared by homogenization and sonication in SDS buffer (Tris-HCl pH 6.8, 5% SDS, 0.2 mM EDTA). Protein quantification was performed using the Pierce BCA Protein Assay Kit. Samples were mixed in 2×Tricine-SDS sample buffer, boiled and separated with Novex™ 16% Tricine Protein Gels. Samples were then transferred onto nitrocellulose membranes, blocked with milk for 60 min at room temperature and probed overnight with anti-CD59 (1/500; Santa Cruz Biotechnology, sc-133170), anti-Emm (1:10) or anti-actin (1:1000; Cell Signaling, #5125) diluted in PBST containing 5% BSA. Immune complexes were revealed with anti-human IgG HRP (Abliance) or anti-mouse HRP (Jackson ImmunoResearch Laboratories) using a chemiluminescence kit (Clarity Western ECL Substrate, Bio-Rad).

Immunoprecipitation of CD59

RBC ghosts from Emm-positive and Emm-negative subjects were incubated overnight at 4° C. with a mouse monoclonal anti-CD59 antibody (1:50; BD Pharmagen) in PBS/BSA. Samples were lysed in lysis buffer (50 mM Tris-HCl pH 8, 150 mM NaCl, 0.5% Igepal, 0.25% sodium deoxycholic acid and 0.0025% SDS) for 60 min on ice, and the lysates were cleared by centrifugation (15 000 g for 15 min at 4° C.). Immune complexes were purified with UltraLink Immobilized Protein A/G (Pierce), and the immunoprecipitated proteins were eluted in 2×Tricine-SDS sample buffer at 95° C. for 5 min and analyzed by western blot as described above.

Disruption of PIGA, PIGG, PIGN, PIGO, PIGS and MPPE1 in K562 by CRISPR/Cas9

Gene editing by CRISPR/Cas9 technology was performed as previously described^{12,13}. The pSpCas9(BB)-2A-Puro (PX459) expression vector was purchased from Addgene (plasmid #48139). For each gene three RNA guides were selected with CRISPRdirect: http://crispr.dbcls.jp/¹⁴. Oligonucleotides were ligated into the BbsI linearized PX459 plasmid. The integrity of each cloned guide sequence was checked by Sanger sequencing with PX459 plasmid sequencing primer. The resultant plasmids were purified with NucleoBond Xtra Midi Plus EF (Macheray-Nagel). Five micrograms of plasmid were electroporated with 10⁶ K-562 cells using Nucleofector II (kit V—program T-016—Lonza). Cells were seeded at a density of 150, 000 cells per mL 48 hours before electroporation. Viability was >95% on the day of transfection. Transfected K562 cells were grown with puromycin (3-5 μg/mL during 3-4 days) in the culture medium 24 hours after transfection until selection were done. Then cells were grown without puromycin. Generation of INDEL events in the targeted exon was assessed using T7 endonuclease I enzyme (New England Biolabs) 15 days after transfection. The K562 WT cells correspond to K562 cells transfected with Cas9 nuclease alone without guide RNA-guide.

Plasmid Construction and Cell Transfection

Human PIGG WT cDNA was obtained in the pcDNA3.1(+)-C-eGFP vector (provided by GeneScript) and subcloned into the pCEP4 vector (Invitrogen). To obtain PIGG H216Y mutant cDNA, an Agilent QuikChange site-directed mutagenesis kit was used according to the manufacturer's instructions.

K562 PIGG KO cells were transfected with 5 μg of pCEP4-Empty, pCEP4-hPIGG^{WT} or pCEP4-hPIGG^{H}214Y using Amaxa® Cell Line Nucleofector® Kit V (Lonza) according to the manufacturer's instructions. Stable transfectants were obtained after 10 days of selection with hygromycin B (0.2 mg/mL, Invitrogen).

Lipid Adsorption

Thawed RBCs from Emm-positive and Emm-negative subjects were incubated for 120 min with 0.5 mg of porcine brain polar lipid extract (Avanti) in PBS containing 1% methanol. After 3 washes in PBS, the surface level of the Emm antigen was analyzed by flow cytometry.

Results

PIGG Underlies the Emm Blood Group

To identify the molecular basis of the Emm blood group antigen, whole-exome sequencing was performed in genomic DNA from three unrelated Emm-negative non-PNH probands (P1, P2 and P3). Variant-filtering strategies led to the identification of three mutations in the common gene PIGG (GenBank: NM_001127178). Proband 1 carried a homozygous variant, c.640C>T; (p. His214Tyr) (data not shown), which is absent from public and in-house databases (Imagine Institute, Polyweb). Proband 2 was homozygous for a G deletion at the +1 position of the splice donor site of intron 5 (c.901+1delG) (data not shown). The splice mutation has been reported in gnomAD only in the heterozygous state, with an allele frequency of 9/248268. Proband 3 carries a 4 kb deletion that removed exon 6 (data not shown). PIGG encodes a 983-amino acid protein, a GPI ethanolamine phosphate (EtNP) transferase 2 (also known as phosphatidylinositol glycan anchor biosynthesis, class G), that is involved in the addition of a side chain modification on the second mannose of GPI¹⁵. Flow cytometry analysis of Emm-negative RBCs with a specific anti-Emm antibody eluate confirmed the absence of the Emm antigen in PIGG-mutated RBCs, while GPI-AP CD59 was normally expressed (data not shown). Next, to validate that Emm expression is controlled by PIGG, we used the CRISPR-Cas9 approach to inactivate this gene in K562 cells. Notably, flow cytometry analysis revealed a strong decrease in Emm expression in PIGG knockout cells but normal expression of CD59 (data not shown). In addition, PIGG cDNA bearing the p.His214Tyr variant found in the P1 proband could not rescue the surface abundance of Emm in these cells, whereas the overexpression of wild-type (WT) PIGG could (data not shown). Although GPI biosynthesis is carried out on the ER membrane, immunofluorescence staining of nonpermeabilized WT and PIGG-deficient cells confirmed the cell surface expression of the Emm antigen (data not shown). These results show that the PIGG gene underlies the Emm blood group antigen.

Emm Antigen Corresponds to Free GPIs

Since the Emm antigen is not expressed in PNH type III cells, which are caused by somatic null mutations in the PIGA gene², we examined Emm expression in PIGA-deficient K562 cells. As expected, these cells lost CD59 and the Emm antigen (data not shown), confirming that Emm expression is related to GPI. Next, to address whether the Emm antigen is carried by the GPI backbone or by a protein attached to GPI, we inactivated PIGS, a major protein of the GPI-transamidase complex responsible for GPI attachment to proteins¹⁶. Interestingly, flow cytometric analysis showed that knockout of PIGS in K562 cells greatly increased Emm expression and, as expected, abolished CD59 expression (data not shown). Accordingly, immunofluorescence staining of permeabilized cells showed that the subcellular localization of Emm was shifted to the cell surface after PIGS inactivation (data not shown), which indicates that Emm is not carried by a protein attached to GPI.

To confirm this finding, we then used an anti-Emm antibody for western blot analysis of the generated KO K562 cell lines. Interestingly, no specific band corresponding to GPI-AP was detected in the 180-to-10 kDa region, but a specific band appeared below the 10-kDa marker in WT K562 cells but not in PIGG and PIGA KO cells (data not shown). In accordance with the flow cytometry data, the intensity of the Emm band was enhanced in PIGS KO cells (data not shown). These results indicate that the Emm antigen is carried by unlinked GPI (free GPI), which was further supported by the increase in Emm labeling of Emm-negative and control RBCs upon incubation with polar lipid extracts presumably containing unlinked GPI glycolipid (data not shown). In addition, since PIGG underlies Emm expression, we conclude that anti-Emm recognizes EtNPs on the second mannose of free GPI. However, this EtNP transferred by PIGG was removed by the phosphodiesterase PGAP5 (encoded by the MPPE1 gene) after the attachment of GPI to proteins and is not present in the most mature GPI-APs (data not shown)⁹. To determine whether the presence of EtNP2 in GPI-AP leads to an increase in Emm expression (data not shown), we generated PGAP5-deficient cells (MPPE1 KO cells) and analyzed them with anti-Emm. Flow cytometry and immunofluorescence analysis showed that PGAP5 inactivation does not increase the expression of Emm, although GPI-AP is normally expressed (data not shown). Normal expression of CD59 and Emm was confirmed by western blot analysis, which also indicates that the migration profile of the Emm band remains unchanged (data not shown).

Finally, immunoprecipitation of CD59 from Emm-negative and control RBCs was performed. As expected, the Emm antigen was not coprecipitated with CD59 from Emm-positive RBCs (data not shown). All these data showed that anti-Emm does not recognize the second EtNP in GPI-APs and confirmed that the Emm antigen is carried by free GPI.

The Emm Epitope is Composed of the Second and Third EtNPs of the Free GPI

The core backbone of GPI is formed by three EtNPs, three mannoses (Man), one non-N-acetylated glucosamine, and inositol phospholipids. Each mannose is modified by one EtNP group. The transfer of EtNPs to Man1, Man2 and Man3 is catalyzed by three EtNP transferases, PIGN, PIGG and PIGO, respectively (data not shown)^{15,17,18}. To determine if EtNP1 and/or EtNP3, in addition to EtNP2, are also parts of the free GPI epitope recognized by anti-Emm, we generated PIGN and PIGO KO cells. Flow cytometric analysis showed that knockout of PIGN in WT K562 cells weakly increased cell surface anti-Emm staining, while inactivation of PIGO completely abolished the expression of Emm (data not shown). Flow cytometry results were confirmed by western blot analysis of the cell lysates with anti-Emm (data not shown). Overall, we demonstrate that the Emm epitope is composed of the second and third EtNPs of the free GPI.

The Surface Expression of Emm Antigen is Altered in RBCs from IGD Patients

Importantly, the expression level of the Emm antigen is under the control of several genes involved in GPI biosynthesis, suggesting that loss-of-function mutations in PIG genes could be associated with a weak Emm blood phenotype. This point is very important for analyzing cells from patients with IGD caused by germline mutations in at least 21 PIG genes. In IGD patients, the expression level of GPI-APs in blood cells was often not indicative of the gene defect and did not correlate with the severity of the clinical phenotype¹⁹. This finding is confirmed by the normal expression of CD59 (data not shown) in RBCs from the P1 proband even though he suffered from intellectual disability and hypotonia¹ potentially caused by the pathogenic His214Tyr mutation in PIGG (data not shown). By contrast, the Emm-negative phenotype (data not shown) indicated a defect in GPI biosynthesis. Thus, we decided to investigate whether pathogenic mutations in other PIG genes involved in IGDs are associated with low expression of the Emm antigen on the RBC surface. IGD patients were clinically investigated at the Necker Hospital in Paris and selected from the Exome database of the Imagine Institute via the Polyweb interface (data not shown). The PIGN patient was a female infant who died suddenly from a severe epileptic seizure at the age of 4. She had a global developmental delay and suffered from intractable epilepsy, severe hypotonia and muscular atrophy. Exome sequencing identified a pathogenic mutation c.284G>A (p. Arg95Gln) in the PIGN gene (data not shown). This mutation has been described previously in patients with MCAHS²⁰. Flow cytometry analysis of her RBCs showed a normal expression level of CD59 and a slight increase in Emm expression compared to that of control RBCs (data not shown). The increase of Emm expression in these RBCs is consistent with PIGN-deficient K562 cell line (data not shown). The PIGO patient is a 10-year-old son of Egyptian consanguineous parents who suffers from intellectual disability, cerebral atrophy, hypotonia and delayed psychomotor development (data not shown). The c.23T>C (p. Leu8Pro) variant in PIGO has been reported in another IGD patient (ClinVar database, rs755191263). Interestingly, flow cytometry analysis of RBCs from this patient showed a strong decrease in Emm expression, while CD59 expression was marginally reduced compared to that of an unrelated control (data not shown). Finally, the PIGA patient is a 6-year-old male who suffers from refractory epilepsy and a severe developmental delay, including delays in language development and motor ability; he is bedridden and does not make eye contact (data not shown). This patient carries a maternal pathogenic mutation, c.241C>T (p.Arg81Cys), which was reported in another patient with MCAHS²¹. Flow cytometry analysis showed that the germline mutation c.241C>T in PIGA does not severely impact Emm and CD59 expression in mature RBCs, although this mutation is responsible for the aberrant development of this patient (data not shown). Collectively, the clinical features resulting from pathogenic variants in PIGG and PIGO genes were corroborated by the altered expression of Emm in RBCs, while GPI-AP expression was not altered.

### Discussion

The major finding in this study is that free, unlinked GPI is expressed at the RBC surface and carries the Emm blood antigen. To date, the GPI is involved in the expression of six other blood group systems including YT, DO, CROM, JMH, CD59, and KANNO^{2,22,23}. However, Emm is the only antigen carried by the GPI backbone and not by a protein linked to GPI. Although the GPI is synthesized in the ER, the presence of several GPI precursors at the cell surface has been documented in mammalian cells and parasites such as Toxoplasma gondii and Plasmodium falciparum^{24,25}. The Plasmodium-free GPI is highly immunogenic and elicits a parasite-specific IgG response in people living in malaria endemic areas²⁵. Consistently, our newly characterized anti-free GPI, anti-Emm, was often described as a naturally-occurring antibody in all Emm-negative patients. In addition, anti-Emm recognizes the GPI precursor that contains three EtNPs but only the second and third EtNP are included in the epitope. The second EtNP, transferred by PIGG, is a transient side-chain that is removed by PGAP5 and that is absent from the mature GPI-APs (data not shown)⁹. The normal surface expression of Emm in PGAP5-defective cells is consistent with the fact that anti-Emm is specific for free GPI. Overall, we provide the unequivocal demonstration that free GPIs are expressed at the human red cell surface and are normal components of cell membranes. Although almost nothing is known about the physiological roles of the free GPIs in human cells, we have recently reported a pathological effect of the free GPIs in PNH patient with PIGT mutations⁵. PIGT-deficiency results on an abnormal accumulation of the unlinked free GPIs that enhances the activation of NLRP3 inflammasome and complement. Although both PIGA- and PIGT-PNH are characterized by GPI-AP deficiency, they display different molecular alterations. Like PIGS, PIGT is a GPI transamidase involved in the attachment of GPI to proteins in the ER¹⁶. PIGA is required for the first step in GPI biosynthesis; therefore, no GPI precursor is generated in PIGA-defective cells (data not shown). Consistently, the Emm antigen (free GPI) is highly expressed in PIGS KO cells (data not shown) but is absent in PIGA-PNH RBCs². Overall, anti-Emm appears as a new helpful tool to detect GPI defect and Emm phenotyping could be associated to GPI-AP analyzing for the diagnosis of PNH disease.

Defective biosynthesis of the GPI is caused by several different genetic mechanisms. In PNH disease, the GPI deficiency is caused by somatic null or nearly null mutations in PIGA that occur in the hematopoietic stem cells (HSCs). By contrast, germline mutations in PIGA and other PIG genes cause IGD²⁶. Null germline mutations in PIGA are thought to be embryonic lethal²⁷, suggesting that p.Arg81Cys found in our patient has residual function (data not shown). Consistent with previous findings²⁸, CD59 expression in RBCs from our PIGA-IGD patient is not strongly affected, which is in accordance with the absence of intravascular hemolysis and erythroid disorders. The GPI-AP defect in IGD patients is often more conspicuous on granulocytes than in erythrocytes, suggesting that the consequence of germline mutations in PIG genes is tissue-specific. In line with this finding, many differences in GPI structure were found among different mammalian tissues and cell lines, suggesting that the GPI anchor may be regulated in a tissue-specific way. In fact, the most prominent clinical symptoms of IGD are neurological ones, including seizures, developmental delay/intellectual disability, cerebral atrophy and hypotonia¹¹. Presumably, the partial reduction of GPI-APs is less tolerated in neuronal development than hematopoiesis as demonstrated in a hiPSC model²⁹. In other hand, the homozygous null alleles of PIGG found in Emm-negative patients do not cause embryonic lethality, but do cause IGD (data not shown). Interestingly, these mutations result on total absence of a free GPI with unaltered GPI-AP expression in both RBCs and PIGG KO cells (data not shown). In addition, we showed that GPI defect in PIGO-IGD patient is also detected by the altered expression of Emm in RBCs, while GPI-AP expression was not altered. These findings are consistent with the implication of both PIGG and PIGO genes in the epitope structure of Emm (data not shown). Further analyses in IGD patients with other PIG mutations are needed to validate the relevance of Emm expression at RBC surface in this pathology.

In conclusion, we provide both genetic and cellular evidence that the Emm antigen is carried by free GPI at the RBC surface and establish Emm as a novel human blood group system. The natural occurrence of anti-Emm is consistent with the high immunogenicity of free GPI reported in numerous microorganisms^{30,31} and suggests a potential role of this antibody in protection against some infectious agents. These findings have important implications for understanding the biological function of human free GPI and the intracellular trafficking of GPI. A pilot study in IGD and PNH patients would be necessary to evaluate the clinical relevance of an Emm phenotyping approach compared to GPI-AP expression studies.

### REFERENCES

Throughout this application, various references describe the state of the art to which this invention pertains. The disclosures of these references are hereby incorporated by reference into the present disclosure.
1. Daniels G L, Taliano V, Klein M T, McCreary J. Emm. A red cell antigen of very high frequency. Transfusion. 1987; 27(4):319-321.
2. Telen M J, Rosse W F, Parker C J, Moulds M K, Moulds J J. Evidence that several high-frequency human blood group antigens reside on phosphatidylinositol-linked erythrocyte membrane proteins. Blood. 1990; 75(7):1404-1407.
3. Takeda J, Miyata T, Kawagoe K, et al. Deficiency of the GPI anchor caused by a somatic mutation of the PIG-A gene in paroxysmal nocturnal hemoglobinuria. Cell. 1993; 73(4):703-711.
4. Krawitz P M, Hochsmann B, Murakami Y, et al. A case of paroxysmal nocturnal hemoglobinuria caused by a germline mutation and a somatic mutation in PIGT. Blood.
2013; 122(7):1312-1315.
5. Hochsmann B, Murakami Y, Osato M, et al. Complement and inflammasome overactivation mediates paroxysmal nocturnal hemoglobinuria with autoinflammation. J Clin Invest. 2019; 129(12):5123-5136.
6. Kinoshita T. Biosynthesis and biology of mammalian GPI-anchored proteins. Open Biol. 2020; 10(3):190290.
7. Wang Y, Maeda Y, Liu Y S, et al. Cross-talks of glycosylphosphatidylinositol biosynthesis with glycosphingolipid biosynthesis and E R-associated degradation. Nat Commun. 2020; 11(1):860.
8. Satpute-Krishnan P, Ajinkya M, Bhat S, Itakura E, Hegde R S, Lippincott-Schwartz J. ER stress-induced clearance of misfolded GPI-anchored proteins via the secretory pathway. Cell. 2014; 158(3):522-533.
9. Fujita M, Maeda Y, Ra M, Yamaguchi Y, Taguchi R, Kinoshita T. GPI glycan remodeling by PGAP5 regulates transport of GPI-anchored proteins from the E R to the Golgi. Cell. 2009; 139(2):352-365.
10. Wang Y, Hirata T, Maeda Y, Murakami Y, Fujita M, Kinoshita T. Free, unlinked glycosylphosphatidylinositols on mammalian cell surfaces revisited. J Biol Chem. 2019; 294(13):5038-5049.
11. Bellai-Dussault K, Nguyen T T M, Baratang N V, Jimenez-Cruz D A, Campeau P M. Clinical variability in inherited glycosylphosphatidylinositol deficiency disorders. Clin Genet. 2019; 95(1):112-121.
12. Yien Y Y, Ducamp S, van der Vorm L N, et al. Mutation in human CLPX elevates levels of delta-aminolevulinate synthase and protoporphyrin IX to promote erythropoietic protoporphyria. Proc Natl Acad Sci USA. 2017; 114(38):E8045-E8052.
13. Ran F A, Hsu P D, Wright J, Agarwala V, Scott D A, Zhang F. Genome engineering using the CRISPR-Cas9 system. Nat Protoc. 2013; 8(11):2281-2308.
14. Naito Y, Hino K, Bono H, Ui-Tei K. CRISPRdirect: software for designing CRISPR/Cas guide RNA with reduced off-target sites. Bioinformatics. 2015; 31(7):1120-1123.
15. Makrythanasis P, Kato M, Zaki M S, et al. Pathogenic Variants in PIGG Cause Intellectual Disability with Seizures and Hypotonia. Am J Hum Genet. 2016; 98(4):615-626.
16. Ohishi K, Inoue N, Kinoshita T. PIG-S and PIG-T, essential for GPI anchor attachment to proteins, form a complex with GAA1 and GPI8. EMBO J. 2001; 20(15):4088-4098.
17. Hong Y, Maeda Y, Watanabe R, et al. Pig-n, a mammalian homologue of yeast Mcd4p, is involved in transferring phosphoethanolamine to the first mannose of the glycosylphosphatidylinositol. J Biol Chem. 1999; 274(49):35099-35106.
18. Hong Y, Maeda Y, Watanabe R, Inoue N, Ohishi K, Kinoshita T. Requirement of PIG-F and PIG-O for transferring phosphoethanolamine to the third mannose in glycosylphosphatidylinositol. J Biol Chem. 2000; 275(27):20911-20919.
19. Knaus A, Pantel J T, Pendziwiat M, et al. Characterization of glycosylphosphatidylinositol biosynthesis defects by clinical features, flow cytometry, and automated image analysis. Genome Med. 2018; 10(1):3.
20. Thiffault I, Zuccarelli B, Welsh H, et al. Hypotonia and intellectual disability without dysmorphic features in a patient with PIGN-related disease. BMC Med Genet. 2017; 18(1):124.
21. Jiao X, Xue J, Gong P, et al. Analyzing clinical and genetic characteristics of a cohort with multiple congenital anomalies-hypotonia-seizures syndrome (MCAHS). Orphanet J Rare Dis. 2020; 15(1):78.
22. Anliker M, von Zabern I, Hochsmann B, et al. A new blood group antigen is defined by anti-CD59, detected in a CD59-deficient patient. Transfusion. 2014; 54(7):1817-1822.
23. Omae Y, Ito S, Takeuchi M, et al. Integrative genome analysis identified the KANNO blood group antigen as prion protein. Transfusion. 2019; 59(7):2429-2435.
24. Ferguson M A. The structure, biosynthesis and functions of glycosylphosphatidylinositol anchors, and the contributions of trypanosome research. J Cell Sci. 1999; 112 (Pt 17):2799-2809.
25. Naik R S, Branch O H, Woods A S, et al. Glycosylphosphatidylinositol anchors of Plasmodium falciparum: molecular characterization and naturally elicited antibody response that may provide immunity to malaria pathogenesis. J Exp Med. 2000; 192(11):1563-1576.
26. Johnston J J, Gropman A L, Sapp J C, et al. The phenotype of a germline mutation in PIGA: the gene somatically mutated in paroxysmal nocturnal hemoglobinuria. Am J Hum Genet. 2012; 90(2):295-300.
27. Nozaki M, Ohishi K, Yamada N, Kinoshita T, Nagy A, Takeda J. Developmental abnormalities of glycosylphosphatidylinositol-anchor-deficient embryos revealed by Cre/loxP system. Lab Invest. 1999; 79(3):293-299.
28. Tarailo-Graovac M, Sinclair G, Stockler-Ipsiroglu S, et al. The genotypic and phenotypic spectrum of PIGA deficiency. Orphanet J Rare Dis. 2015; 10:23.
29. Yuan X, Li Z, Baines A C, et al. A hypomorphic PIGA gene mutation causes severe defects in neuron development and susceptibility to complement-mediated toxicity in a human iPSC model. PLoS One. 2017; 12(4):e0174074.
30. Striepen B, Zinecker C F, Damm J B, et al. Molecular structure of the “low molecular weight antigen” of Toxoplasma gondii: a glucose alpha 1-4 N-acetylgalactosamine makes free glycosyl-phosphatidylinositols highly immunogenic. J Mol Biol. 1997; 266(4):797-813.
31. Tomavo S, Couvreur G, Leriche M A, et al. Immunolocalization and characterization of the low molecular weight antigen (4-5 kDa) of Toxoplasma gondii that elicits an early IgM response upon primary infection. Parasitology. 1994; 108 (Pt 2):139-145.

1. (canceled)

2. A method of diagnosing an inherited glycosylphosphatidylinositol (GPI) deficiency in a subject and treating the subject, comprising
detecting expression of Emm antigen in a sample of red blood cells obtained from the subject and
administering a Histone deacetylase (HDCA) inhibitor to the subject identified as having an alteration of the expression of the Emm antigen.

3. The method of claim 2 wherein the subject suffers from intellectual disability and/or epilepsy.

4. The method of claim 2 wherein expression of GPI-anchored proteins is not decreased in the red blood cells of the subject.

5. The method of claim 2 wherein expression of at least one GPI-AP in the blood cells does not indicate the GPI deficiency of the subject.

6. The method of claim 5 wherein the at least one GPI-AP is selected from the group consisting of CD59, CD55, FLAER, and CD16.

7. The method of claim 2 wherein the subject suffers from paroxysmal nocturnal hemoglobinuria.

8. The method of claim 3, wherein the subject suffers from epilepsy and the method further comprises a step of administering an anti-epileptic drug to the subject.
